(19) United States
(12) Patent Application Publication
(10) Pub. No.: US 2009/0070900 A1
(43) Pub. Date: Mar. 12, 2009

(54) Novel cherkasky materials and novel use of biomolecules and biomasses

(76) Inventor: Alexander Cherkasky, Duesseldorf (DE)

Correspondence Address: Alexander Cherkasky, Prinz-Georg Str. 5, Duesseldorf, 40477, DE

(21) Appl. No.: 12/155,688

(22) Filed: Jun. 9, 2008

(30) Foreign Application Priority Data
Jun. 12, 2007 (DE) 102007027596.1
Jun. 13, 2007 (DE) 102007027241.5

(51) Int. Cl. C09K 3/00; C08K 5/00; C08L 1/00; C08L 5/00; C08K 5/16; C08L 89/00; C12N 1/12; C12N 1/14; C12N 1/20; C12N 5/04; C08K 11/00; A01H 5/00; A01H 11/00

(52) U.S. Cl. 800/298; 252/182.12; 252/182.3; 524/9; 524/17; 524/27; 524/35; 524/58; 524/115; 524/174; 524/186; 435/255.2; 435/257.1; 435/254.1; 435/252.1; 435/252.8; 800/295

(57) ABSTRACT

Novel materials containing dispersed or aligned biomolecules and/or cells and biomasses, which are preferably chemically modified, can be preferably used as conductors, semiconductors, transistors, resistors or non-conductors and as artificial jewels and gemologic materials, materials for use in computer, aerospace, marine and automobile industries, as well as modified alloys for machines and pipelines, boring and drilling machines and materials, and for pumps and for oil industry, as well as for furniture, for wood paper and cellulose industry, as well as for textile industry.

### FIELD OF THE INVENTION

The invention relates to the fields of composite materials, composite biomaterials, bionics, biology, technical biology, chemistry, biochemistry, materials design, material science and engineering.

### BACKGROUND OF THE INVENTION

Composite materials with proteins and composite biomaterials are known, including: composite materials from calcium salts and proteins (Knopf et al EP1676555(A2) 2006, Jul. 5), as well as “Composite biomaterials comprising calcium phosphate materials, collagen and glycosaminoglycans” (Lynn Andrew and Cameron Ruth EP (A1) 2006, Aug. 16) and “Composite materials for bone implants” (Gonzalez Santos Raman and Enzare Paz Alberto Guillermo EP 1679090 (A1) 2006, Jul. 12). Various composition materials and their uses are not elaborated.

The present invention comprises novel composite materials with novel uses.

### DESCRIPTION OF THE INVENTION

The invention comprises novel materials and composite materials that have additional advantages and more advantages in physical and chemical properties and live more, i.e. remain more time without microdeformations.

The novel materials according to the present invention contain chemically modified biomolecules, and/or cells or biomasses, which are dispersed or aligned on form of fibres.

The chemical modification can include addition of halogens F, Cl, Br, I preferably through the reaction with HF, HCl, HBr and HI or with elementary gases or liquid F₂, Cl₂, Br₂ or I₂. The chemical modification may also include complexing or addition of Ge, As, Te, Se, Si, B or P.

The chemically modified biomolecules may include amino acids alanine, leucine, isoleucine, proline, phenylalanine, tryptophan, methionine, glycine, serine, threonine, cystein, tyrosine, asparagine, glutamine, asparagic acid, glutamic acid, lysine, arginine, histidine, nucleotides, thymine, uracile, cytosine, guanine, adenine, ATP, lipids, sphingolipids, phosholipids, anthocyans, isoprenes, chlorophylls, peptides, proteins, nucleic acids RNA and DNA, mono, oligo and polysaccharides.

The peptides and proteins may include neuropeptides and hormones such as NPY, galanin, leptin, orexins A and B, substance P, enkephalins, endorphins as well as serum bovine albumin, keratins, collagens, alpha, beta, gamma crystallins, silk fibroin, lamprin, abducin, fibrin, fibrinogen, tenascin, thrombospondin, titin, neuroproteins, Myelin Basic Protein (MBP), Glial fibrillary acidic protein (GFAP), immunoglobulines, storage proteins such as gliadines, vicilines, binding proteins such as lectins, PP2 from Cucurbitas, Cip A cellulose binding protein, phloem proteins such as PP1 from Cucurbitas, metal binding proteins coeruloplasmin, ferritin, astacin.

The saccharides can be selected from cellulose, hemicellulose, pectin, chitin, chitosan, hyaloronic acid.

The composite material according to the present invention can also possess or contain other substances and objects for example seeds, corns and grains.

The organelles may include mitochondria, chloroplasts, nuclei, nucleoli, liposomes and peroxisomes.

The cells and biomasses may include alga such as Volvox, Chlamydomas, fungi, yeast such as Saccharomyces, Pichia, lichens such as Cladonia, bacteria such as Escherischia coli, mosses such as Sphagnum and plants such as Elodea.

The material, that contains the above-listed biomolecules, cells and other substances, may include metals Li, Me, Na Mg, Al, K, Ca, Sc, Ti, V, Cr, Mn, Fe, Co, Ni, Cu, Zn, Ga, Rb, Sr, Y, Zr, Nb, Mo, Ru, Rh, Pd, Ag, Cd, In, Sn, Sb, Cs, Ba, La, Pr, Nd, Sm, Eu, Gd, Tb, Dy, Ho, Er, Tm, Yb, Lu, Hf, Ta, W, Re, Os, Ir, Pt, Au, Hg, Tl, Pb, Bi, half-metal Ge, non metals B, C, Si, P, Te, Se, As. The material containing the chemically modified molecules and/or organelles and/or cells or other substances and objects may also be a combination of elements and include alloys, ceramics, gemologic substances as well as organic polymers, plastics, biocompatible materials, lignin and timber.

Organic polymers may include plexiglas and polyethylene. Other examples for materials containing chemically modified biomolecules and/or cells include ZrSi, BN, MgCl₂, CaO, GeO, GaO, SiO₂.

The concentration of chemically modified or non-modified molecules and cells in materials may vary from preferably 0.01 to 98 percent.

The chemically modified or non modified molecules and cells can be used as additives to materials for ennobling of artificial jewels and gemologic materials, for improvement of physical, optical, chemical properties of materials, as well as to make materials long living. This may be explained with the replacement of hubs in materials through biomolecules or cells, when materials or more exactly their molecular structures are considered as networks of atoms and molecules according to The Theory of Networks of Laszlo Barabasi. Biomolecules and/or cells, also in small concentrations (0.01-5 percent) may replace hubs in molecular structures and thus prolong life of materials and improve their properties. The advantages may be also based on the action of dispersed substances as nano- and microelements in molecular structures. The materials and composite materials with dispersed biomolecules and/or cells, according to this invention can be used as conductors and semiconductors, and transistors, resistors for example in solar batteries, computer chips, computers, microprocessors, in computer, aerospace, marine and automobile industries. The materials according to the present invention may also be used for or in pipelines, boring and drilling machines, for filters, for pumps and for oil industry as well as for furniture, for wood, paper, cellulose, chemical and textile industries. These materials according to the present invention may also be used as absorption materials, building materials, as artificial jewels and gemologic materials such as perles, corals and diamonds, as well as additives to lubricants, building materials, labelling materials, dyes, cosmetics, perfumes, as well as cleansers and detergents. The materials according to this invention, can be produced by addition of chemically modified molecules and/or cells, and/or non modified molecules and/or cells into heated mass for example into heated silicium. Molecules and cells will be dispersed in cooled mass. As result, silicium with dispersed biomolecules and/or cells, arises, which may be either non modified or chemically modified preferably iodated, chlorated, flourated or bromated.

This composite material especially, silicium with dispersed chemically modified biomolecules can be used in electronic devices, especially in computers, chips, microprocessors as well as in solar batteries.

### EXAMPLES

### Example 1

### Silicium with Dispersed Fluorated Serum Bovine Albumine

Commercially obtained serum bovine albumine will be treated with 1% HF. After fluoration, fluorated proteins and amino acids will be added to hot silium mass and mixed. Cooled silicium contains dispersed fluorated proteins and fluorated amino acids.

The silicium with dispersed fluorated proteins and fluorated amino acids can be used in microprocessors, chips, computers, calculators and solar batteries.

Examples 2, 3 and 4 comprise the same method as in example 1, wherein HCl, HI, HBr will be used and silicium contains dispersed chlorated, iodated or bromated proteins and amino acids.

### Example 5

### Silicium with Dispersed Chemically Modified Piomolecules and Biomasses

In hot silicium the mixture will be added containing fluorated algal cells Volvox globator and Chlamydomonas spec. (treated with HF), chlorated bovine albumines and amino acids (treated with HCl), iodated RNA, DNA and nucleotides from Elodea spec. (treated with HI), bromated isoprenes, bromated anthocyanes and selen. Resulting silicium contains dispersed mixture.

The first advantage of the resulting silicium with dispersed substances is that silicium will be saved. The resulting “composite silicium” is long-living and remains his physical and chemical properties.

What is clamed is:

1. A material or composite material containing chemically modified biomolecules and/or organelles, cells and biomasses in preferably dispersed or fibre form, wherein:
a. a material can be selected from, but not limited to the following:
a1. a metal: Li, Be, Na, Mg, Al, K, Ca, Sc, Ti, V, Cr, Mn, Fe, Co, Ni, Cu, Zn, Ga, Rb, Sr, Y, Zr, Nb, Mo, Ru, Rh, Pd, Ag, Cd, In, Sn, Sb, Cs, Ba, La, Ce, Pr, Nd, Sm, Eu, Gd, Tb, Dy, Ho, Er, Tm, Yb, Lu, Hf, Ta, W, Re, Os, Ir, Pt, Au, Hg, Tl, Pb, Bi,
a2. a half-metal: Si, Ge,
a3. a non-metal: B, C, Se, P, Te, As,
a4. any combinations of elements or especially alloys, gemologic substances and ceramics including: ZrSi, BN, MgCl₂, CaO,
a5. organic polymers: polyethylene, plexiglas and plastics,
a6. biocompatible materials, biomaterials, textiles, leather, cotton, lignin, timber, cellulose,
b. chemical modification can be selected from but not limited to: addition of halogens F, Cl, Br, I preferably through reaction with HF, HCl, HBr, and HI or through treatment with liquid or gaseous F₂, Cl₂, Br, I₂ as well as addition or complexing with Ge, As, Te, Se, Si, B and P,
c. biomolecules can be selected from but not limited to:
c1. amino acids: alanine, leucine, isoleucine, proline, phenylalanine, tryptophan, methionine glycine, serine, thereonine, cystein, tyrosine, asparagine, glutamine, asparagic acid, glutamic acid, lysine, arginine, histidine,
c2. nucleotides: guanine, adenine, thymine, uracil, cytosine,
c3. lipids,
c4. anthocyans
c5. isoprenes,
c6. chlorophylls,
c7. peptides and proteins: neuropeptides NPY, galanin, leptin, orexin, insulin, keratin, collagen, crystallin,
c8. polysaccharides: chitin, chitosan, cellulose, hyaloronic acid,
c9. mono- and oligosaccarides,
c10. nucleic acids RNA and DNA,
c11. ATP,
d. organelles, cells and biomasses can be selected but not limited from:
d1. organelles: mitochondria, chloroplasts,
d2. alga: Volvox spec., Chlamydomonas spec, other green alga as well as brown and red alga,
d3. fungi: yeast Saccharomyces spec., Pichia spec.,
d4. lichens: Cladonia,
d5. bacteria: E. coli,
d6. mosses: Sphagnum spec.,
d7. plants: Elodea spec.

2. Use of a material according to claim 1, as semiconductor, conductor, microprocessors, transistors, resistors, a material preferably in solar batteries, non-conductor, artificial jewels, composite materials, implants and as other material.

3. A material containing biomolecules and/or cells wherein, a material and biomolecules and/or cells can be selected from those listed in claim 1.

4. Use of material to claim 3 as conductors, jewels or gemologic materials, additives in resistors and transistors.

5. Use of either chemically modified or non-modified biomolecules and cells listed in claim 1, as additives to materials, for ennobling of artificial jewels and gemologic materials, for improvement of physical, optical, chemical properties of materials.

6. A method for producing of materials to claim 1, wherein
a. the content of biomolecules and/or cells preferably ranges from 0.01% to 98%,
b. biomolecules or cells will be added to heated material mass
